(19) United States
(12) Patent Application Publication
(10) Pub. No.: US 2016/0310109 A1
(43) Pub. Date: Oct. 27, 2016

(54) ULTRASONIC IMAGING APPARATUS AND CONTROL METHOD THEREFOR

(71) Applicant: SAMSUNG ELECTRONICS CO., LTD., Suwon-si (KR)

(72) Inventors: Sung Chan Park, Gyeonggi-do (KR); Joo Young Kang, Gyeonggi-do (KR); Kyu Hong Kim, Seoul (KR); Jung Ho Kim, Gyeonggi-do (KR); Su Hyun Park, Gyeonggi-do (KR)

(21) Appl. No.: 15/102,253

(22) PCT Filed: Dec. 5, 2014
(86) PCT No.: PCT/KR2014/011967

§ 371 Date: Jun. 6, 2016

(30) Foreign Application Priority Data
Dec. 6, 2013 (KR) 10-2013-0151336

(51) Int. Cl. A61B 8/08; G01S 7/52; G06T 5/00; A61B 8/00

(52) U.S. Cl. CPC A61B 8/5269; A61B 8/5207; A61B 8/461; A61B 8/54; G01S 7/52085; G06T 5/003; G06T 2207/10132; G06T 2207/30004

(57) ABSTRACT

Disclosed herein is an ultrasonic imaging apparatus. The ultrasonic imaging apparatus includes an image restoration unit to perform image restoration on at least one beamformed ultrasound image, an image restoration performance estimation unit to estimate an image restoration performance based on the beamformed ultrasound image and setting information regarding ultrasound image acquisition, and an adaptive postprocessing unit to perform adaptive postprocessing on a result image acquired by the image restoration based on the estimated image restoration performance and thus resolution of a restored image and signal-to-noise ratio (SNR) may be enhanced.

### TECHNICAL FIELD

Embodiments of the present invention relate to an ultrasonic imaging apparatus to generate an image of the interior of an object using ultrasonic waves and a control method therefor.

### BACKGROUND ART

Ultrasonic imaging apparatuses are imaging apparatuses that collect internal information of an object (e.g., a human body) using ultrasonic waves and acquire an image of the interior of the object using the collected information.

More particularly, an ultrasonic imaging apparatus may collect ultrasonic waves reflected or generated from a target site inside an object and acquire a cross-sectional image of various tissues, structures or the like inside the object, e.g., a cross-sectional image of various organs, soft tissues, or the like, using the collected ultrasonic waves. To implement such operation, the ultrasonic imaging apparatus may direct ultrasonic waves to a target site inside an object from the outside to collect ultrasonic waves reflected from the target site inside the object.

Ultrasonic imaging apparatuses may generate ultrasonic waves of a predetermined frequency using ultrasonic transducers or the like, direct the ultrasonic waves of a predetermined frequency to a target site, and receive ultrasonic waves reflected from the target site, thereby acquiring ultrasound signals of a plurality of channels corresponding to the received ultrasonic waves. Such ultrasonic imaging apparatuses correct time differences between ultrasound signals of a plurality of channels and focus the ultrasound signals to obtain beamformed ultrasound signals, and generate and acquire an ultrasound image using the beamformed ultrasound signals so that a user can view a cross-sectional image of the interior of an object.

Such ultrasonic imaging apparatuses are smaller in size and less expensive than other apparatuses, exhibit real-time display of an image of the interior of an object, and have no risk of exposure to radiation such as X-rays, and thus are widely used in a variety of fields, such as medicine and the like.

### DISCLOSURE OF INVENTION

### Technical Problem

Therefore, it is an aspect of the present invention to provide an ultrasonic imaging apparatus that composes images using an adaptive postprocessing technique based on image restoration performance (e.g., resolution and noise variance) estimated for a restored image and thus may enhance resolution and signal-to-noise ratio (SNR) of the restored image and a control method therefor.

It is another aspect of the present invention to provide an ultrasonic imaging apparatus that composes images using an adaptive postprocessing technique based on image restoration performance (e.g., resolution and noise variance) estimated for a plurality of restored images and thus may enhance contrast of the restored images and a control method therefor.

Additional aspects of the invention will be set forth in part in the description which follows and, in part, will be obvious from the description, or may be learned by practice of the invention.

### Solution to Problem

In accordance with one aspect of the present invention, an ultrasonic imaging apparatus includes an image restoration unit to perform image restoration on at least one beamformed ultrasound image, an image restoration performance estimation unit to estimate an image restoration performance based on the beamformed ultrasound image and setting information regarding ultrasound image acquisition, and an adaptive postprocessing unit to perform adaptive postprocessing on a result image acquired by the image restoration based on the estimated image restoration performance.

The setting information may be at least one of an image restoration parameter and a time gain compensation (TGC).

The image restoration performance estimation unit may estimate the image restoration performance according to depth of a target site inside an object.

The image restoration performance estimation unit may estimate the image restoration performance according to a region of a target site inside an object.

The image restoration unit may include a point spread function estimation unit to estimate a point spread function for the beamformed ultrasound image and a deconvolution unit to perform image restoration on the beamformed ultrasound image based on the estimated point spread function.

The adaptive postprocessing may be a noise attenuation process to reduce noise increased in the deconvolution unit.

The adaptive postprocessing unit may be any one of an anti-aliasing filter or a speckle reduction filter.

The ultrasonic imaging apparatus may further include a display unit to display a result image acquired by the adaptive postprocessing.

In accordance with another aspect of the present invention, a method of controlling an ultrasonic imaging apparatus includes performing image restoration on at least one beamformed ultrasound image, estimating an image restoration performance based on the beamformed ultrasound image and setting information regarding ultrasound image acquisition, and performing adaptive postprocessing on a result image acquired by the image restoration based on the estimated image restoration performance.

The setting information may be at least one of an image restoration parameter and a time gain compensation (TGC).

The estimating may include estimating the image restoration performance according to depth of a target site inside an object.

The estimating may include estimating the image restoration performance according to a region of a target site inside an object.

The performing of the image restoration may include estimating a point spread function for the beamformed ultrasound image and performing deconvolution on the beamformed ultrasound image based on the estimated point spread function.

The adaptive postprocessing may be a noise attenuation process to reduce noise increased during the deconvolution.

The adaptive postprocessing may be performed using an anti-aliasing filter or a speckle reduction filter.

The method may further include displaying a result image acquired by the adaptive postprocessing.

In accordance with another aspect of the present invention, an ultrasonic imaging apparatus includes an image restoration unit to perform image restoration on at least one beamformed ultrasound image using images having different speckle patterns, an image restoration performance estimation unit to estimate an image restoration performance based on the beamformed ultrasound image and setting information regarding ultrasound image acquisition, and an adaptive postproces sing unit to perform adaptive postprocessing on a result image acquired by the image restoration based on the estimated image restoration performance.

### Advantageous Effects of Invention

According to the ultrasonic imaging apparatus and the control method therefor as described above, the following effects can be obtained.

By composing images using an adaptive postprocessing technique based on image restoration performance (e.g., resolution and noise variance) estimated for a restored image, it is possible to enhance resolution and signal-to-noise ratio (SNR) of the restored image.

By composing images using an adaptive postprocessing technique based on image restoration performance (e.g., resolution and noise variance) estimated for a plurality of restored images, it is possible to enhance contrast of the restored images.

### BRIEF DESCRIPTION OF DRAWINGS

These and/or other aspects of the invention will become apparent and more readily appreciated from the following description of the embodiments, taken in conjunction with the accompanying drawings of which:

FIG. 1 is a perspective view illustrating an exterior appearance of an ultrasonic imaging apparatus according to an embodiment of the present invention;

FIG. 2 is a control block diagram of the ultrasonic imaging apparatus according to the embodiment illustrated in FIG. 1;

FIG. 3 is a plan view of an ultrasonic probe illustrated in FIG. 2;

FIG. 4 is a specific configuration view of a beamforming unit illustrated in FIG. 2;

FIG. 5 is a configuration view for explaining an image restoration unit;

FIG. 6 is a conceptual view for explaining a point spread function;

FIG. 7 is a view for explaining a relationship between an ideal image and a radio frequency (RF) image and deconvolution;

FIG. 8A illustrates images for explaining a relationship between an ideal image and an RF image;

FIG. 8B illustrates an example of an RF signal-based ultrasound image of a target site according to depth;

FIG. 8C is an ultrasound image for explaining a depth of a target site;

FIG. 9 is a configuration view particularly illustrating an image restoration unit illustrated in FIG. 2;

FIGS. 10 and 11 are views illustrating a method of applying adaptive postprocessing of a plurality of restored images; and

FIG. 12 is a flowchart illustrating an ultrasonic imaging apparatus control method according to an embodiment of the present invention.

### BEST MODE FOR CARRYING OUT THE INVENTION

Reference will now be made in detail to the embodiments of the present invention, examples of which are illustrated in the accompanying drawings, wherein like reference numerals refer to like elements throughout.

Hereinafter, embodiments of the present invention will be described in detail with reference to the accompanying drawings.

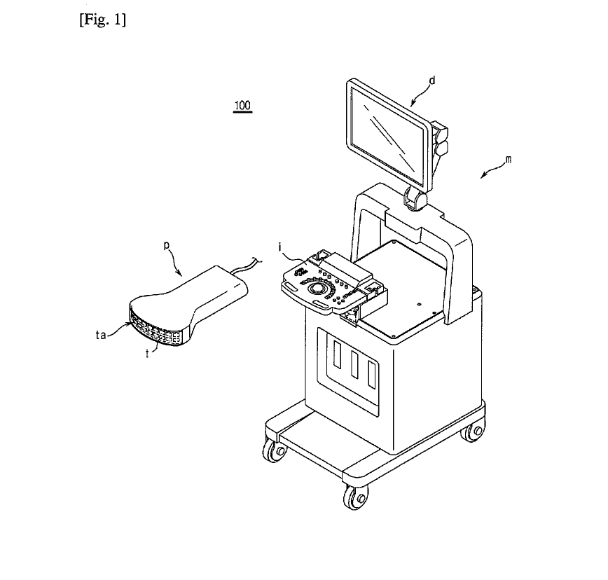

FIG. 1 is a perspective view illustrating an exterior appearance of an ultrasonic imaging apparatus 100 according to an embodiment of the present invention.

Ultrasonic imaging apparatuses are imaging apparatuses that transmit ultrasonic waves to a target site inside an object, e.g., a human body from a surface of the human body, receive ultrasonic waves (ultrasonic echo waves) reflected from the target site, and generate a cross-sectional image of various tissues or structures inside the object using information regarding the received ultrasonic waves. As illustrated in FIG. 1, the ultrasonic imaging apparatus 100 may include an ultrasonic probe p to transmit ultrasonic waves to an object, to receive ultrasonic echo waves from the object, and to convert the received ultrasonic echo waves into an electrical signal, i.e., an ultrasound signal, and a main body m connected to the ultrasonic probe p and including an input unit i and a display unit d. The ultrasonic probe p is provided at an end portion thereof with an ultrasonic transducer array ta. The ultrasonic transducer array ta means that a plurality of ultrasonic transducers t is arranged in the form of an array. As illustrated in FIG. 1, the ultrasonic transducers t may be arranged in the form of a linear array or a convex array.

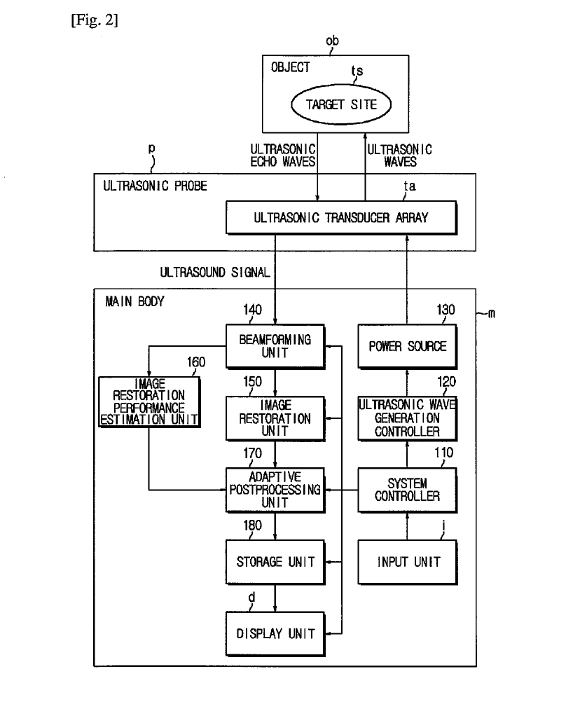

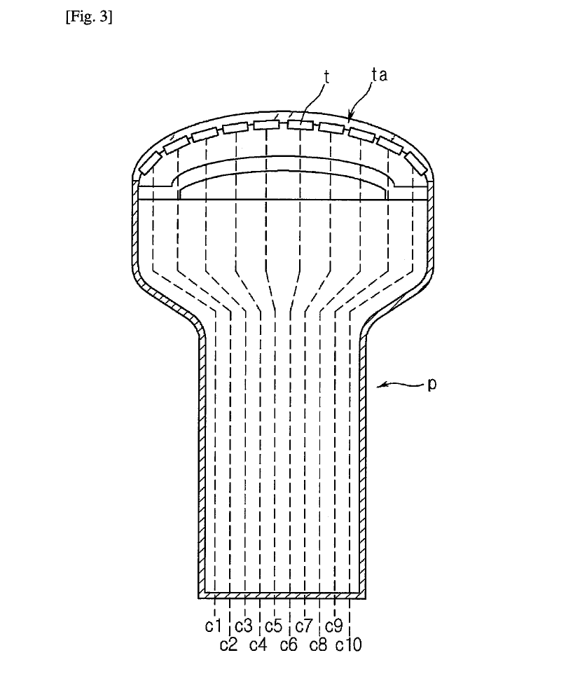

FIG. 2 is a control block diagram of the ultrasonic imaging apparatus 100 according to the embodiment illustrated in FIG. 1. FIG. 3 is a plan view of the ultrasonic probe p illustrated in FIG. 1.

As illustrated in FIGS. 2 and 3, the ultrasonic probe p may include the ultrasonic transducers t to generate ultrasonic waves according to a voltage (or current) applied, to transmit the generated ultrasonic waves to at least one target site ts inside an object ob, to receive ultrasonic echo waves reflected from the target site ts of the object ob, and to convert the received ultrasonic echo waves into an electrical signal. As illustrated in FIG. 3, the ultrasonic transducer array ta in which the ultrasonic transducers t are arranged in the form of an array may be installed at an end of the ultrasonic probe p. In this case, the ultrasonic transducers t may be disposed at an end of the ultrasonic probe p in at least one column.

A transducer is a device to convert a predetermined type of energy into another type of energy. In this regard, the ultrasonic transducers t may convert electrical energy into wave energy or vice versa. Accordingly, the ultrasonic transducers t may function as both an ultrasonic wave generation element and an ultrasonic wave receiving element.

The ultrasonic transducer array ta vibrates by a pulse signal or alternating current applied to the ultrasonic transducer array ta according to a control signal of a system controller 110 installed at the main body m to generate ultrasonic waves. The generated ultrasonic waves are directed to the target site ts inside the object ob. In this case, the ultrasonic waves generated by the ultrasonic transducer array ta may be directed by focusing on a plurality of target sites ts inside of the object ob. That is, the generated ultrasonic waves may be directed to the target sites ts through multifocusing.

The ultrasonic waves generated by the ultrasonic transducer array ta are reflected from at least one target site ts inside the object ob to return to the ultrasonic transducer array ta. The ultrasonic transducer array ta receives ultrasonic echo waves reflected back from the at least one target site ts. When the ultrasonic echo waves reach the ultrasonic transducer array ta, the ultrasonic transducer array ta vibrates with a predetermined frequency corresponding to a frequency of the ultrasonic echo waves to output alternating current of a frequency corresponding to the vibration frequency of the ultrasonic transducer array ta. Accordingly, the ultrasonic transducer array ta may convert the received ultrasonic echo waves into a predetermined electrical signal.

Each ultrasonic transducer t receives external ultrasonic waves and output an electrical signal into which the ultrasonic waves have been converted and thus, as illustrated in FIG. 4, the ultrasonic probe p may output electrical signals of a plurality of channels c1 to c10. In this case, the number of channels may be, for example, 64 to 128.

The ultrasonic transducer t may include a piezoelectric vibrator or a thin film. When alternating current is supplied to piezoelectric vibrators or thin films of the ultrasonic transducers t from a power source (not shown) such as an external power supply or an internal electrical storage device, e.g., a battery or the like, the piezoelectric vibrators or thin films vibrate with a predetermined frequency according to the applied alternating current and ultrasonic waves of a predetermined frequency are generated according to the vibration frequency. On the other hand, when ultrasonic echo waves of a predetermined frequency reach the piezoelectric vibrators or thin films, the piezoelectric vibrators or thin films vibrate according to the ultrasonic echo waves. In this regard, the piezoelectric vibrators or thin films output alternating current of a frequency corresponding to the vibration frequency.

The ultrasonic transducer t may, for example, be any one of a magnetostrictive ultrasonic transducer using a magnetostrictive effect of a magnetic body, a piezoelectric ultrasonic transducer using a piezoelectric effect of a piezoelectric material, and a capacitive micromachined ultrasonic transducer (cMUT), which transmits and receives ultrasonic waves using vibration of several hundreds or several thousands of micromachined thin films. In addition, other kinds of transducers that may generate ultrasonic waves according to an electrical signal or generate an electrical signal according to ultrasonic waves may also be used as the ultrasonic transducer t.

In addition, the ultrasonic transducer array ta may be in the form of a one-dimensional ultrasonic transducer array in which a plurality of ultrasonic transducers t1 to t10 (see FIG. 4) are arranged one-dimensionally, i.e., in a single row. In another embodiment, the ultrasonic transducer array ta may be in the form of a two-dimensional ultrasonic transducer array in which a plurality of ultrasonic transducers is arranged two-dimensionally, i.e., in a planar form.

As illustrated in FIG. 2, the main body m may include the system controller 110, an ultrasonic wave generation controller 120, a power source 130, a beamforming unit 140, an image restoration unit 150, an image restoration performance estimation unit 160, an adaptive postprocessing unit 170, a storage unit 180, the input unit i, and the display unit d.

The system controller 110 controls overall operations of the main body m. In particular, the system controller 110 may generate a predetermined control signal for each element of the main body m, e.g., the ultrasonic probe p, the ultrasonic wave generation controller 120, the beamforming unit 140, the image restoration unit 150, the adaptive postprocessing unit 170, the storage unit 180, and the display unit d illustrated in FIG. 2. In particular, the system controller 110 implements a control operation so as to calculate a time delay value according to distance differences between convergence points of the ultrasonic transducers t included in the ultrasonic probe p and the object ob, to form transmitting and receiving beams according to the calculated time delay value, and to generate a transmitting and receiving signal according thereto.

In addition, the system controller 110 may control the ultrasonic imaging apparatus 100 by generating a predetermined control command for each element of the main body m according to predetermined setting or according to an instruction or command of a user input via the input unit i.

The ultrasonic wave generation controller 120 may receive predetermined control commands from the system controller 110 or the like, generate predetermined control signals according to the received control commands, and transmit the control signals to the ultrasonic transducer array ta of the ultrasonic probe p. In this case, the ultrasonic transducer array ta may generate ultrasonic waves by operating according to the received predetermined control signals. In addition, the ultrasonic wave generation controller 120 may generate a control signal for the power source 130 electrically connected to the ultrasonic transducer array ta according to the received control command and transmit the generated control signal to the power source 130. In this case, the power source 130 having received the control signal may supply alternating current of a predetermined frequency to the ultrasonic transducer array ta according to the control signal so that the ultrasonic transducer array ta generates ultrasonic waves of a frequency corresponding to the frequency of the alternating current.

The beamforming unit 140 performs beamforming based on the ultrasound signals of the channels c1 to c10 transmitted from the ultrasonic transducer array ta. In this regard, beamforming refers to a process whereby signal intensity is enhanced by super-position of signals when transmitting and receiving signals using a plurality of transducers (converters). That is, the beamforming process focuses a plurality of received signals input via a plurality of channels to acquire an appropriate ultrasound image of the interior of the object ob. A detailed description of configuration and function of the beamforming unit 140 will be provided in the description with reference to FIG. 4.

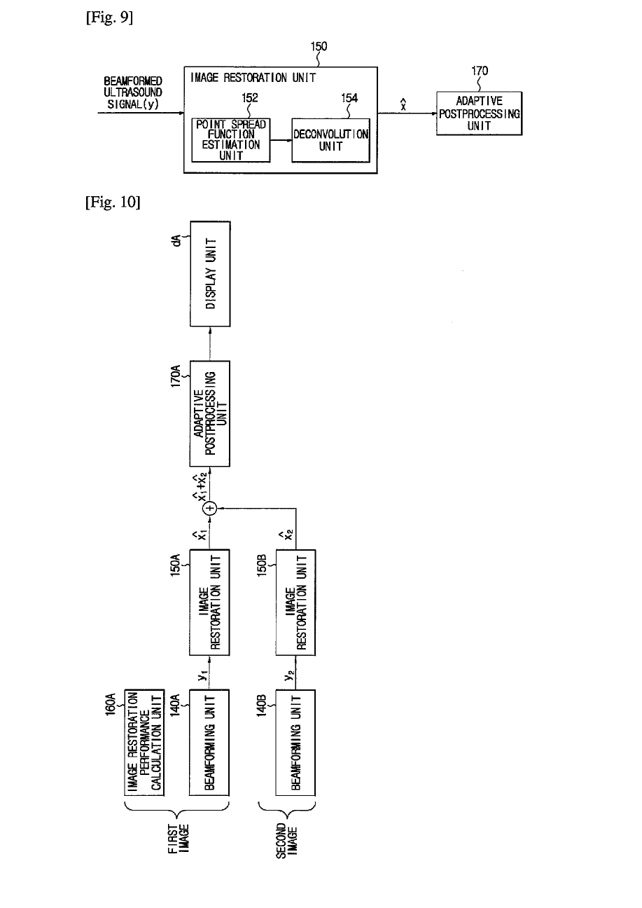

The image restoration unit 150 implements image restoration based on the ultrasound image (an image acquired as a result of beamforming) of the object ob, generated based on the signal focused by the beamforming unit 140. A detailed description of configuration and function of the image restoration unit 150 will be provided in the description with reference to FIG. 9.

The image restoration performance estimation unit 160 estimates (calculates) image restoration performance based on various setting information regarding ultrasound image acquisition input via the input unit i, e.g., signal separation parameters, time gain compensation (TGC) values, and ultrasound signal beamformed (focused) by the beamforming unit 140. In this regard, image restoration performance may, for example, be measurement of resolution of an ultrasound image of a local region using autocorrelation, noise variance using an intensity ratio of autocorrelation to system noise, or the like. In addition, the image restoration performance estimation unit 160 may calculate resolution and noise variance of an ultrasound image according to depth or region of the target site ts. The image restoration performance estimation unit 160 transmits the calculated resolution and noise variance of the ultrasound image to the adaptive postproces sing unit 170.

In the present embodiment, a case in which the image restoration performance estimation unit 160 to estimate image restoration performance based on the setting information input by a user and the ultrasound signal beamformed by the beamforming unit 140 is separately arranged has been described by way of example. However, in another embodiment, the image restoration performance estimation unit 160 may be included in the adaptive postprocessing unit 170.

The adaptive postprocessing unit 170 implements adaptive postprocessing on an image \hat{x} restored by the image restoration unit 150 based on the image restoration performance (e.g., resolution and noise variance of an ultrasound image) transmitted from the image restoration performance estimation unit 160. In this regard, postprocessing may for example be noise reduction (NR) to reduce noise increased in a de-convolution unit 154 (see FIG. 9) included in the image restoration unit 150. The adaptive postprocessing unit 170 may be embodied as an anti-aliasing filter, a speckle reduction filter, or the like.

In addition, log compression, digital scan conversion (DSC), or the like is performed, thereby acquiring a final result image.

The storage unit 180 may temporarily or permanently store the ultrasound image. The ultrasound image stored in the storage unit 180 may be an ultrasound image generated by the image restoration unit 150 or an ultrasound image corrected (postprocessed) by the adaptive postprocessing unit 170.

The input unit i may receive commands related to operations of the ultrasonic imaging apparatus 100, input by a user. Examples of commands input by a user via the input unit i include ultrasonic diagnosis initiation commands, commands to select a mode such as an amplitude mode (A-mode), a brightness mode (B-mode), a motion mode (M-mode), and the like, and various setting information regarding ultrasound image acquisition, e.g., image restoration (deconvolution) parameters, noise boost up control parameters, time gain compensation (TGC), and the like. TGC is a parameter for compensating for attenuation of ultrasonic echo waves according to depth. In this regard, the input unit i may for example be a keyboard, a mouse, a trackball, a tablet, a touch screen module, or the like through which a user can input data, an instruction, or a command.

The display unit d displays ultrasound images acquired during ultrasonic diagnosis and menus or instructions needed for ultrasonic diagnosis. The display unit d may directly display the ultrasound image generated by the image restoration unit 150 to a user or display the ultrasound image having been image processed by the adaptive postprocessing unit 180 to a user. In addition, the display unit d may display the ultrasound image stored in the storage unit 190 to a user. The ultrasound image displayed on the display unit d may be an A-mode ultrasound image, a B-mode ultrasound image, or a 3D stereoscopic ultrasound image. In this regard, the display unit d may for example be a cathode ray tube (CRT), a liquid crystal display (LCD) device, or the like.

FIG. 4 is a view particularly illustrating a structure of the beamforming unit 140 illustrated in FIG. 2.

The beamforming unit 140 installed in the main body m receives the ultrasound signals of the channels c1 to c10 from the ultrasonic transducer array ta, focuses the received ultrasound signals of the channels c1 to c10, and outputs the beamformed ultrasound signal. The beamformed ultrasound signal may form an ultrasound image. In particular, the beamforming unit 140 performs beamforming to estimate the size of reflected waves in a specific space for the ultrasound signals of the channels c1 to c10.

As illustrated in FIG. 4, the beamforming unit 140 may include a time difference correction unit 142 and a focusing unit 144.

The time difference correction unit 142 may correct time differences among ultrasound signals output from the respective ultrasonic transducers t1 to t10.

As described above, the ultrasonic transducer array ta receives ultrasonic echo waves reflected from the target site ts. While distances between each of the ultrasonic transducers t1 to t10 installed at the ultrasonic probe p and the target site ts are different, the sound velocities of ultrasonic waves are nearly constant although they differ according to media. Thus, each of the ultrasonic transducers t1 to t10 receives ultrasonic echo waves generated or reflected from the same target site is at different times. Accordingly, although each of the ultrasonic transducers t1 to t10 receives the same ultrasonic echo waves, predetermined time differences occur between ultrasound signals output from the ultrasonic transducers t1 to t10. The time difference correction unit 142 corrects the time differences between the ultrasound signals output from the ultrasonic transducers t1 to t10.

To correct the time differences between the ultrasound signals, for example, as illustrated in FIG. 4, the time difference correction unit 142 delays transmission of ultrasound signals to be input to particular channels (e.g., the channels c1 to c10) according to predetermined settings to some extent so that the ultrasound signals of the channels c1 to c10 are transmitted to the focusing unit 144 at the same time.

The focusing unit 144 may focus ultrasound signals. As illustrated in FIG. 4, the focusing unit 144 may focus the ultrasound signals of the channels c1 to c10 in which time differences therebetween have been corrected.

The focusing unit 144 may focus ultrasound signals by adding a predetermined weight, e.g., a beamforming coefficient, to each input ultrasound signal to emphasize or relatively attenuate an ultrasound signal at a predetermined location. Accordingly, an ultrasound image according to user needs may be generated.

In one embodiment, the focusing unit 144 may focus ultrasound signals using a predefined beamforming coefficient regardless of the ultrasound signals. In another embodiment, the focusing unit 144 may obtain an appropriate beamforming coefficient based on the input ultrasound signals and focus the ultrasound signals using the obtained beamforming coefficient.

A beamformed ultrasound signal y obtained by the beamforming unit 140 is transmitted to the image restoration unit 150, as illustrated in FIG. 4.

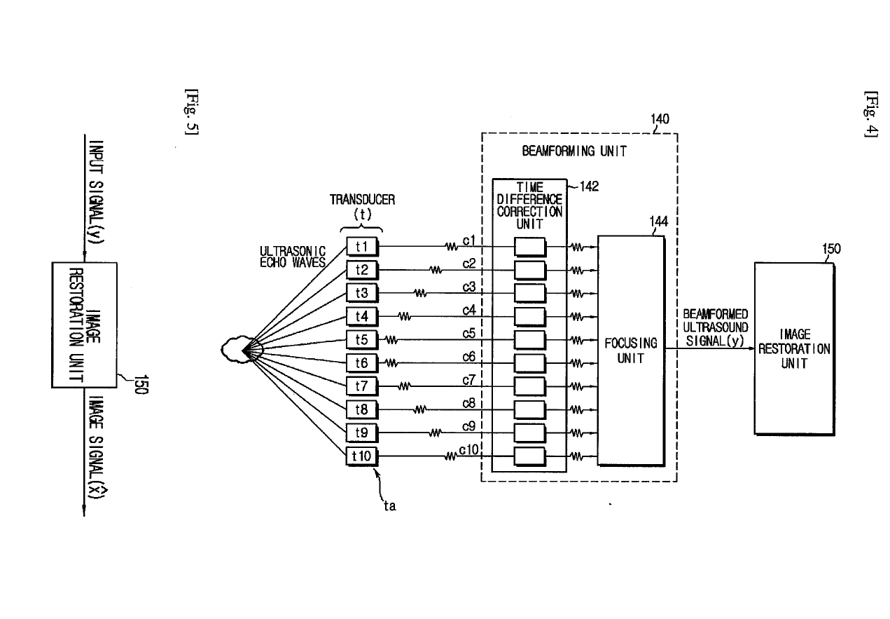

FIG. 5 is a configuration view for explaining the image restoration unit 150.

As illustrated in FIG. 5, the image restoration unit 150 generates an image signal \hat{x} based on an input signal y and outputs the generated image signal \hat{x}. That is, the image restoration unit 150 implements image restoration based on the acquired image data y of the object ob.

The term “image restoration” as used herein refers to scaling of a low-resolution image to a high-resolution image using an estimated point spread function (PSF). That is, image restoration means an operation of enhancing image resolution.

In this regard, the input signal y may be a signal acquired from ultrasonic waves, which are sound waves with an audible frequency of greater than 20 kHz. The image restoration unit 150 generates or acquires an image with a higher resolution than input image data (e.g., the input signal y) by estimating at least one PSF to generate the image signal \hat{x} from the input signal y and performing deconvolution using the estimated results. The image restoration unit 150 outputs the generated or acquired image in the form of the image signal \hat{x}.

The PSF is a function for generation of final image data by combination with image data acquired by photographing of an imaging apparatus and is mainly used to restore ideal image data.

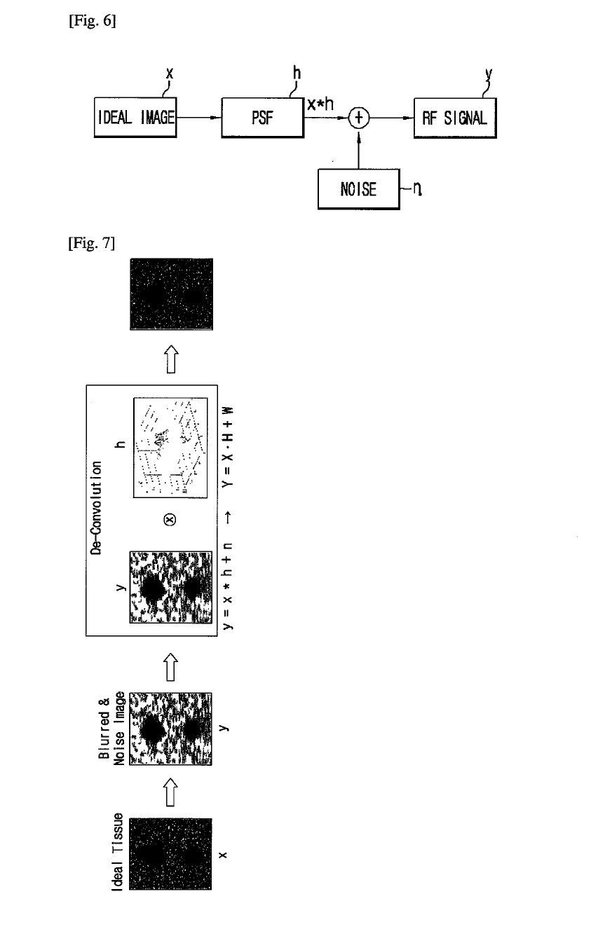

FIG. 6 is a conceptual view for explaining the PSF.

As illustrated in FIG. 6, an imaging apparatus outputs a signal different from an ideal image x, e.g., a radio frequency signal y such as an ultrasound signal generated from an ultrasonic imaging apparatus, or the like, due to technical characteristics or physical characteristics of the imaging apparatus or noise η in a process of acquiring an image of the object ob.

That is, the RF signal y acquired by the imaging apparatus is a signal output by adding the noise η to the ideal image x modified according to technical characteristics or physical characteristics of the imaging apparatus.

FIG. 7 is a view for explaining a relationship between an ideal image and an RF image and deconvolution.

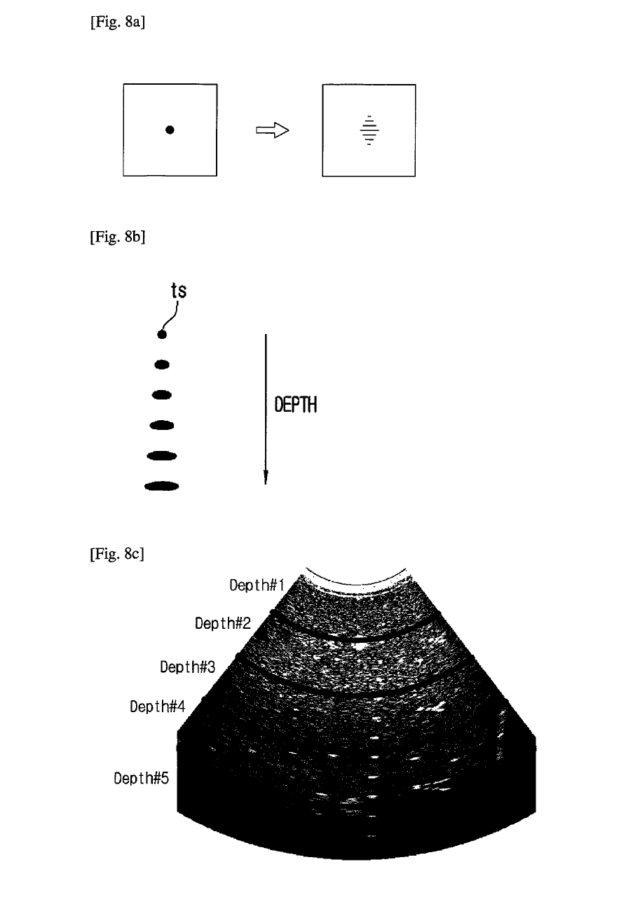

The leftmost image of FIG. 7 shows an ideal shape of a tissue in a human body. When the ideal image is given as x as illustrated in FIG. 7, an ultrasound image collected by the ultrasonic probe p of the ultrasonic imaging apparatus 100 and beamformed is represented by y in the middle portion of FIG. 7. That is, the ideal image x becomes different from an image y acquired from the RF signal. This will be described below in detail with reference to FIGS. 8A to 8C.

FIG. 8A illustrates images for explaining a relationship between an ideal image and an RF image.

FIG. 8A illustrates an input signal-based image as an example of an ultrasound image acquired by an ultrasonic imaging apparatus. When an ideal image x of a target site ts under ideal conditions is represented as a left image illustrated in FIG. 8A, an image based on an input signal y, e.g., an RF signal, of the target site ts is represented as a right image illustrated in FIG. 8A. In particular, the target site ts in the input signal-based image is displayed as if the target site ts in the ideal image x extends upward, downward, leftward, and rightward. That is, the input signal-based image is considerably different from the ideal image x and thus, when the image based on the input signal y, i.e., an RF signal, is restored, the target site ts of the restored image becomes different from the target site ts of the ideal image x.

The ideal image x and the input signal-based image may be different according to depth or the like. FIG. 8B illustrates an example of an RF signal-based ultrasound image of the target site ts according to depth. FIG. 8C illustrates an ultrasound image for explaining the depth of the target site ts.

As illustrated in FIG. 8B, when a distance between the target site ts and an image data collection member, e.g., the ultrasonic probe p, is short, for example, as illustrated in FIG. 8C, when a lesion inside a human body is positioned at a first depth (Depth #1), the input signal-based image of the target site ts is the same or considerably similar to an ideal image of the target site ts. On the other hand, when the distance between an image data collection member and the target site ts is long, for example, when a lesion inside the body is positioned at a fourth depth (Depth #4) or a fifth depth (Depth #5) illustrated in FIG. 8C, the input signal-based image of the target site ts is shown as extending in a lateral direction and thus considerably differs from the ideal image x of the target site ts. That is, the target site ts of the ideal image x and the target site ts of the input signal-based image become further different according to distance between a data collection member and the target site ts.

Thus, when the ideal image x is restored using the RF signal y, a difference between the ideal image x and the image based on the RF signal y needs to be corrected, whereby an accurate image of the target site ts may be acquired. In this case, image restoration is implemented such that, assuming that an original image o and the acquired RF signal y have a predetermined relationship, the RF signal y is corrected using a predetermined function corresponding to the predetermined relationship. In this regard, the predetermined function is a PSF h. Here, the PSF h is a function of brightness distribution obtained from an actual focused surface when point inputs pass through an imaging system.

A relationship among the ideal image x, the PSF h, the noise and the input signal y, i.e., an RF signal, may be represented by Equation 1 below.

y=x*h+n [Equation 1]

wherein y is an RF signal output, h is a PSF, x is a signal for an ideal image, and n denotes noise.

Assuming that there is no noise, the RF signal y may be represented by convolution between a high-resolution image x and the PSF h. Thus, when an appropriate PSF h for the measured RF signal y is identified, the high-resolution image x corresponding to the measured RF signal y may be acquired. In other words, when the PSF h and the RF signal y are identified, a high-resolution image that is the same or almost the same as an object may be restored.

As described above, a process of obtaining the restored image \hat{x} using the PSF h is referred to as deconvolution. After deconvolution, aliasing and noise boost up occur.

A deconvolution model for image restoration of the measured RF signal y may be represented by Equation 2 below.

\hat{x}=min λ(y−x*h)²+|x|^{α}, α=0.5˜2 [Equation 2]

wherein λ is a restoration parameter and α is a value corresponding to norm.

In this regard, when the restoration parameter λ is set to a small value, resolution gain may be deteriorated, while the SNR may be increased. On the other hand, when the restoration parameter λ is set to a large value, resolution gain may be enhanced, while the SNR may be reduced. That is, differences in resolution enhancement may occur according to restoration parameter values.

In addition, differences in image restoration performance may occur according to depth of the target site ts or a plurality of regions constituting the target site ts. For example, aliasing occurs in a region in which changes in sound velocity are less severe, and noise boost up occurs in a region in which signal attenuation is severe.

Thus, in embodiments of the present invention, resolution of the restored image and the SNR may be enhanced by setting the restoration parameter X of the deconvolution model to a large value and preventing reduction in SNR using the adaptive postprocessing unit 170.

In addition, in embodiments of the present invention, resolution of the restored image and the SNR may be enhanced by calculating image restoration performance (e.g., resolution and noise variance of an ultrasound image) according to depth of the target site ts or a plurality of regions constituting the target site ts and performing adaptive postprocessing on the restored image based on the image restoration performance calculated according to the depth or regions.

FIG. 9 is a configuration view particularly illustrating the image restoration unit 150 illustrated in FIG. 2.

The image restoration unit 150 outputs the image signal \hat{x} with high resolution using an RF signal, i.e., the input signal y and a PSF h appropriate for the input signal y, in a direction opposite a direction indicated by an arrow illustrated in FIG. 6. That is, as illustrated in the middle portion of FIG. 7, the image restoration unit 150 generates the restored image \hat{x} with high resolution by performing deconvolution by applying an appropriate PSF h to the RF signal y.

As illustrated in FIG. 9, the image restoration unit 150 may include a PSF estimation unit 152 and the deconvolution unit 154.

In general, blurring in which an image is blurred due to non-focusing of an object ob and an image acquisition apparatus (e.g., an ultrasonic imaging apparatus) occurs in an image acquired by an image data acquisition unit (e.g., a beamforming unit). In this regard, blurring is a phenomenon whereby brightness of peripheral pixels is distorted by a PSF in which brightness of a single pixel of an ideal image represents a degree of blurring. A blurred image may be modeled by convolution of an ideal image and a PSF. When the PSF is identified, restoration of the ideal image from the blurred image is referred to as deconvolution. In general, however, it is difficult to identify a PSF of a blurred image and thus a process of estimating the PSF is needed.

The PSF estimation unit 152 may receive the ultrasound signal y beamformed by the beamforming unit 140 and estimate a PSF h from the beamformed ultrasound signal y. In the PSF estimation unit 152, a method of estimating a PSF from an input image is obvious to those of ordinary skills in the art, and thus, a detailed description thereof will be omitted herein. As an example, US 2002/0049379A1 discloses a method of estimating a PSF from an input image. As another example, to acquire an image that is the same or almost the same as the object ob based on the input signal y through restoration, the PSF estimation unit 152 may estimate a PSF without reduction in resolution from several directions by using a PSF database (not shown) constructed by a one-dimensional (1D) or two-dimensional (2D) PSF, estimating a 2D PSF based on a 1D PSF, or the like.

The deconvolution unit 154 performs image restoration on a degraded image (i.e., the beamformed ultrasound signal y) using the PSF h estimated by the PSF estimation unit 152. That is, the deconvolution unit 154 performs imaging of the object ob using the estimated PSF h and converts the acquired input signal y, e.g., an RF signal into a form or shape that is the same as or similar to form or shape of the original object ob. As illustrated in FIG. 9, the deconvolution unit 154 restores an image of an input signal acquired using the estimated PSF h, i.e., the beamformed ultrasound signal y, and outputs the image signal \hat{x} for the restored image.

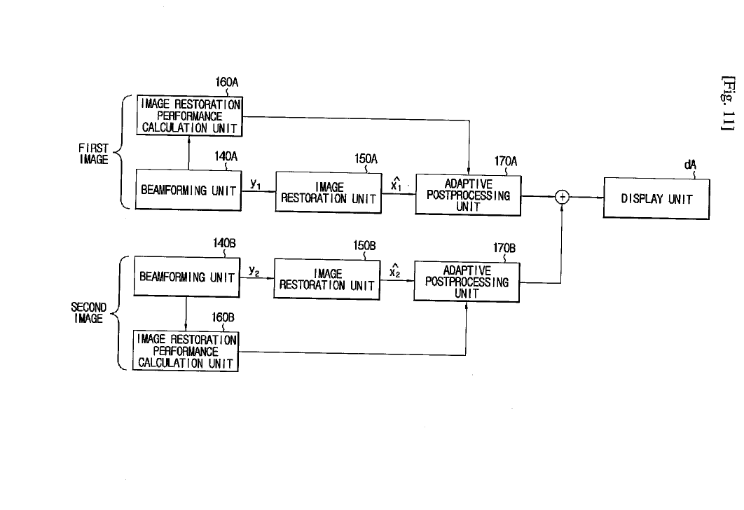

FIG. 10 is a block diagram illustrating a method of applying adaptive postprocessing of a plurality of restored images.

In the present embodiment, a process of performing postprocessing based on a plurality of images will be described.

Ultrasound image compounding is a technique of adding a plurality of images to suppress speckles of the images and enhance only a target site (e.g., a tissue or the like) inside an object. An image having passed through the image restoration unit 150 has excessively increased speckles or noise. These problems may be addressed by ultrasound image compounding.

A compounding technology may be largely divided into angular compounding and frequency compounding. In angular compounding, different images may be generated by varying angles of plane waves in plane wave-based ultrasound synthetic aperture imaging or varying directions of lines in line by line focusing. In frequency compounding, corresponding images may be generated by separating a fundamental frequency and harmonic components upon reception. The generated images may be processed by the adaptive postprocessing unit 170.

A process of performing image processing on a first image is illustrated at an upper side of FIG. 10, and a process of performing image processing on a second image is illustrated at a lower side of FIG. 10.

As illustrated in FIG. 10, an ultrasound signal y₁ acquired by beamforming ultrasound signals for respective channels that constitute a first image among a plurality of images using the beamforming unit 140 is transmitted to the image restoration unit 150. The image restoration unit 150 generates a restored image [symbol] by performing image restoration (deconvolution) based on the beamformed ultrasound signal y₁.

Meanwhile, an ultrasound signal y₂ acquired by beamforming ultrasound signals for respective channels that constitute a second image among the images using the beamforming unit 140 is transmitted to the image restoration unit 150. The image restoration unit 150 generates a restored image [symbol] by performing image restoration (deconvolution) based on the beamformed ultrasound signal y₂.

The restored image [symbol] for the first image and the restored image [symbol] for the second image are compounded, and a compounded restored image is [symbol] + [symbol] is transmitted to the adaptive postprocessing unit 170. The adaptive postprocessing unit 170 performs adaptive postproces sing on the compounded restored image [symbol] + [symbol] based on the image restoration performance (e.g., resolution and noise variance of an ultrasound image) estimated by the image restoration performance estimation unit 160 and transmits the postprocessed restored image to the display unit d.

FIG. 11 is a block diagram illustrating another example of a method of performing adaptive postproces sing on a plurality of restored images.

A process of performing image processing on a first image is illustrated at an upper side of FIG. 11, and a process of performing image processing on a second image is illustrated at a lower side of FIG. 11.

As illustrated in FIG. 11, an ultrasound signal y₁ acquired by beamforming ultrasound signals for respective channels that constitute a first image among a plurality of images using the beamforming unit 140 is transmitted to the image restoration unit 150. The image restoration unit 150 generates a restored image [symbol] by performing image restoration (deconvolution) based on the beamformed ultrasound signal y₁. The image restoration unit 150 transmits the generated restored image [symbol] to the adaptive postproces sing unit 170. The adaptive postproces sing unit 170 performs adaptive postprocessing on the restored image [symbol] based on the image restoration performance (e.g., resolution and noise variance of an ultrasound image) estimated by the image restoration performance estimation unit 160.

Meanwhile, an ultrasound signal y₂ acquired by beamforming ultrasound signals for respective channels that constitute a second image among the images using the beamforming unit 140 is transmitted to the image restoration unit 150. The image restoration unit 150 generates a restored image [symbol] by performing image restoration (deconvolution) based on the beamformed ultrasound signal y₂. The image restoration unit 150 transmits the generated restored image [symbol] to the adaptive postprocessing unit 170. The adaptive postprocessing unit 170 performs adaptive postprocessing on the restored image [symbol] based on the image restoration performance (e.g., resolution and noise variance of an ultrasound image) estimated by the image restoration performance estimation unit 160. The signals on which adaptive postprocessing has been performed are compounded and the compounded signal is transmitted to the display unit d.

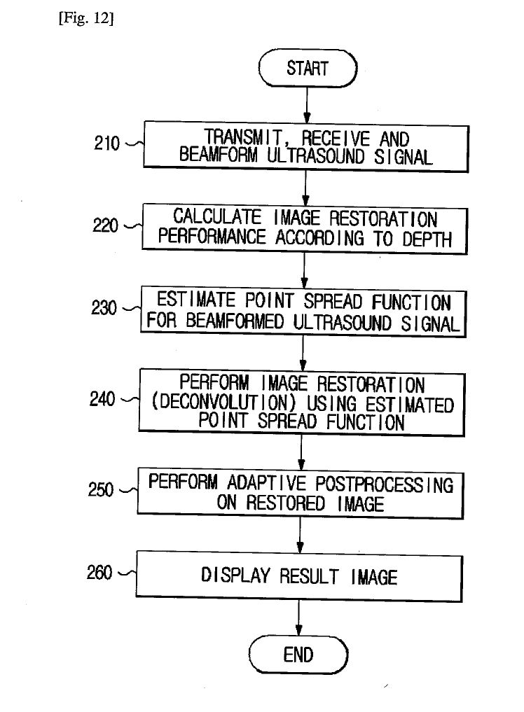

FIG. 12 is a flowchart illustrating an ultrasonic imaging apparatus control method according to an embodiment of the present invention.

First, the system controller 110 controls the ultrasonic wave generation controller 120 and the beamforming unit 140 to perform transmittance and reception of ultrasound signals and beamforming thereof according to initiation commands for ultrasonic diagnosis input via the input unit i. More particularly, the system controller 110 controls the ultrasonic transducer array ta to transmit ultrasonic waves to an object ob by transmitting a control signal to the ultrasonic wave generation controller 120. Subsequently, the ultrasonic transducer array ta receives ultrasonic echo waves reflected back from a surface of the object ob. In this regard, the received ultrasonic echo waves are converted into an electrical signal, i.e., an ultrasound signal and the obtained ultrasound signal is output. When the ultrasonic echo waves are received by the ultrasonic transducers t1 to t10, ultrasound signals of the channels c1 to c10 may be output from the ultrasonic transducers t1 to t10. Time differences between the output ultrasound signals of the channels c1 to c10 are corrected by the correction unit 142 of the beamforming unit 140, and the ultrasound signals, time differences of which have been corrected, are focused by the focusing unit 144 of the beamforming unit 140. As a result, the beamformed ultrasound signal is output.

Next, the image restoration performance estimation unit 160 estimates (calculates) image restoration performance based on various setting information regarding ultrasound image acquisition input via the input unit i, e.g., image restoration parameters, time gain compensation (TGC) values, and ultrasound signals beamformed (focused) by the beamforming unit 140 (operation 220). In this regard, the image restoration performance may, for example, be resolution and noise variance of an ultrasound image, and the like. In addition, the image restoration performance estimation unit 160 may calculate resolution and noise variance of a restored ultrasound image according to depth of a target site is or regions thereof. The image restoration performance estimation unit 160 transmits the calculated resolution and noise variance of the ultrasound image to the adaptive postprocessing unit 170.

That is, resolution and noise variance may be calculated by analyzing results of the restored ultrasound image. Calculation of resolution and noise variance may be performed by auto correlation.

Subsequently, the PSF estimation unit 152 of the image restoration unit 150 receives the ultrasound signal y beamformed by the beamforming unit 140 and estimates a PSF h from the beamformed ultrasound signal y (operation 230).

Next, the deconvolution unit 154 of the image restoration unit 150 performs image restoration (deconvolution) on a degraded image (i.e., the beamformed ultrasound signal y) using the PSF h estimated by the PSF estimation unit 152 (operation 240). That is, the deconvolution unit 154 performs imaging of the object ob using the estimated PSF h, converts the acquired input signal y, e.g., an RF signal into a high-resolution image so as to be viewed as a form or shape that is the same as or similar to form or shape of the original object ob, and outputs an image signal \hat{x} for the restored image.

Next, the adaptive postprocessing unit 170 performs adaptive postprocessing on the image \hat{x} restored by the image restoration unit 150 based on the image restoration performance (e.g., resolution and noise variance of an ultrasound image) transmitted from the image restoration performance estimation unit 160 (operation 250). In this regard, a postprocessing process, for example, noise reduction (NR) for reducing noise increased in the deconvolution unit 154, or the like may be performed. In addition, log compression, digital scan conversion (DSC), and the like are performed to acquire a final result image.

The ultrasound image of the target site is processed by the adaptive postprocessing unit 170 is displayed on the display unit d under control of the system controller 110 (operation 260). Thereby, diagnosis of the object ob using an ultrasound image is completed.

As is apparent from the above description, according to an ultrasonic imaging apparatus and a control method therefor according to embodiments of the present invention, adaptive postprocessing based on image restoration performance (e.g., resolution or noise variance) estimated for a restored image is applied and thus resolution of the restored image and signal-to-noise ratio (SNR) may be enhanced.

In addition, according to an ultrasonic imaging apparatus and a control method therefor according to embodiments of the present invention, images are compounded based on adaptive postprocessing based on image restoration performance (e.g., resolution or noise variance) estimated for a plurality of restored images and thus contrast of the restored images may be enhanced.

Although a few embodiments of the present invention have been shown and described, it would be appreciated by those skilled in the art that changes may be made in these embodiments without departing from the principles and spirit of the invention, the scope of which is defined in the claims and their equivalents.

1. An ultrasonic imaging apparatus comprising:
an image restoration unit to perform image restoration on at least one beamformed ultrasound image;
an image restoration performance estimation unit to estimate an image restoration performance based on the beamformed ultrasound image and setting information regarding ultrasound image acquisition; and
an adaptive postprocessing unit to perform adaptive postprocessing on a result image acquired by the image restoration based on the estimated image restoration performance.

2. The ultrasonic imaging apparatus according to claim 1, wherein the setting information is at least one of an image restoration parameter and a time gain compensation (TGC).

3. The ultrasonic imaging apparatus according to claim 1, wherein the image restoration performance estimation unit estimates the image restoration performance according to depth of a target site inside an object.

4. The ultrasonic imaging apparatus according to claim 1, wherein the image restoration performance estimation unit estimates the image restoration performance according to a region of a target site inside an object.

5. The ultrasonic imaging apparatus according to claim 1, wherein the image restoration unit comprises:
a point spread function estimation unit to estimate a point spread function for the beamformed ultrasound image; and
a deconvolution unit to perform image restoration on the beamformed ultrasound image based on the estimated point spread function.

6. The ultrasonic imaging apparatus according to claim 5, wherein the adaptive postprocessing is a noise attenuation process to reduce noise increased in the deconvolution unit.

7. The ultrasonic imaging apparatus according to claim 6, wherein the adaptive postprocessing unit is any one of an anti-aliasing filter or a speckle reduction filter.

8. The ultrasonic imaging apparatus according to claim 1, further comprising a display unit to display a result image acquired by the adaptive postprocessing. comprising:
performing image restoration on at least one beamformed ultrasound image;
estimating an image restoration performance based on the beamformed ultrasound image and setting information regarding ultrasound image acquisition; and
performing adaptive postprocessing on a result image acquired by the image restoration based on the estimated image restoration performance.

10. The method according to claim 9, wherein the setting information is at least one of an image restoration parameter and a time gain compensation (TGC).

11. The method according to claim 9, wherein the estimating comprises estimating the image restoration performance according to depth of a target site inside an object.

12. The method according to claim 9, wherein the estimating comprises estimating the image restoration performance according to a region of a target site inside an object.

13. The method according to claim 9, wherein the performing of the image restoration comprises:
estimating a point spread function for the beamformed ultrasound image; and
performing deconvolution on the beamformed ultrasound image based on the estimated point spread function.

14. The method according to claim 13, wherein the adaptive postprocessing is a noise attenuation process to reduce noise increased during the deconvolution.

15. The method according to claim 14, wherein the adaptive postprocessing is performed using an anti-aliasing filter or a speckle reduction filter.

16. The method according to claim 9, further comprising displaying a result image acquired by the adaptive postproces sing.

17. An ultrasonic imaging apparatus comprising:
an image restoration unit to perform image restoration on at least one beamformed ultrasound image using images having different speckle patterns;
an image restoration performance estimation unit to estimate an image restoration performance based on the beamformed ultrasound image and setting information regarding ultrasound image acquisition; and
an adaptive postprocessing unit to perform adaptive postprocessing on a result image acquired by the image restoration based on the estimated image restoration performance.
